(19) United States
(12) Patent Application Publication
(10) Pub. No.: US 2003/0165897 A1
(43) Pub. Date: Sep. 4, 2003

(54) Dispatched polypeptides

(76) Inventors: Richard E. Burke, Victoria (AU); Konrad Basler, Kusnacht (CH)

Correspondence Address: MARSHALL, GERSTEIN & BORUN, 6300 SEARS TOWER, 233 SOUTH WACKER, CHICAGO, IL, 60606-6357, US

(21) Appl. No.: 10/168,428

(22) PCT Filed: Dec. 19, 2000
(86) PCT No.: PCT/CH00/00672

(30) Foreign Application Priority Data
Dec. 21, 1999 (US) 09468237

(51) Int. Cl. C12Q 1/68; C07H 21/04; C12P 21/02; C12N 5/06; C07K 14/72; C07K 14/47

(52) U.S. Cl. 435/6; 435/69.1; 435/455; 435/320.1; 435/325; 530/350; 536/23.5

(57) ABSTRACT

The present invention relates to a new family of sterol sensing domain proteins which modulate the release of Hedgehog proteins and lipid modified hydrophobic proteins from cells. Such proteins e.g. are involved in developmental processes, differentiation, regeneration and cell homeostasis, making them particularly suitable tools for diagnosis and therapy.

### CROSS REFERENCE TO RELATED APPLICATION

This application claims the priority of U.S. patent application 09/468,237, filed Dec. 21, 1999 the disclosure of which is incorporated herein by reference in its entirety

### FIELD OF THE INVENTION

The present invention relates to a new family of sterol sensing domain proteins which modulate the release of Hedgehog proteins and lipid modified proteins from cells.

### BACKGROUND OF THE INVENTION

Pattern formation is the activity by which embryonic cells form ordered spatial arrangements of differentiated tissues. The physical complexity of higher organisms arises during embryogenesis through the interplay of cell intrinsic lineage and cell extrinsic signaling. Inductive interactions play an important role in embryonic patterning from the earliest establishment of the body plan to the patterning of the organ systems, to the generation of diverse cell types during tissue differentiation (Davidson E Devel. 108: 365-89, 1990; Gurdon J. B, Cell 68:185-99, 1992; Jessel T. M et al., Cell 68:257-70, 1992). The effects of developmental cell interactions are varied. Typically, responding cells are diverted from one route of cell differentiation to another by inducing cells that differ from both the uninduced and induced states of the responding cells (induction). Cell interactions in early development may be sequential, such that an initial induction between two cell types leads to a progressive amplification of diversity. Inductive interactions occur not only in embryos, but in adult cells as well, and can act to establish and maintain morphogenetic patterns as well as induce differentiation (Gurdon J. B, Cell 68:185-99, 1992).

Members of the Hedgehog (Hh) family of secreted signaling proteins function as potent short range organizers in animal development. The family consists of Drosophila hedgehog and the vertebrate proteins Desert hedgehog, Sonic hedgehog and Indian hedgehog. In the case of Drosophila limb development the short range inducer Hedgehog controls the expression of the long range morphogens Wingless (wg), and Decapentaplegic (dpp) (Basler and Struhl, Nature 368:208. 14, 1994). Each leg and wing primordium is subdivided into two cell populations, the anterior (A) and Posterior (P) compartments. Essential to correct wing patterning is the restriction of Hh signaling activity to a narrow band of A cells. The short range nature of Hh signaling appears to rely on at least two unprecedented mechanisms of receptor circuitry and ligang biosynthesis. The Hh signaling is transduced by a receptor complex consisting of the two cell surface proteins Patched (Ptc) and Smoothened (Smo) (Ingham, EMBO J. 17:3505-11, 1998). Ptc is expressed in all A compartment cells and, in the absence of Hh, inhibits the activity of Smo which is essential for Hh signal transduction. Binding of Hh to Ptc releases latent Smo activity, activating the transduction pathway (Chen and Struhl, Development 125:4943-8, 1998; Murone et al., Current Biology 9:76-84, 1999). A universal response to the Hh signaling in all systems examined so far is the upregulation of ptc transcription (Forbes et al., Development-Supplement, 115-24, 1993; Goodrich et al., Genes & Development 10:301-12, 1996; Marigo et al., Development 122:1225-33, 1996). The accumulation of Ptc protein in Hh-responding wing cells sequesters Hh protein and thereby restricts the further movement of Hh into the A compartment (Chen and Struhl, Cell 87:553-63, 1996).

The second mechanism by which Hh movement is impeded has been elucidated in detailed biochemical studies investigating the processing of Hh to its active signaling form. Hh undergoes an autoproteolytic cleavage reaction to give rise to its active N-terminal portion (Lee et al., Science 266:1528-37, 1994; Porter et al., Nature 374:363-6, 1995). This cleavage is accompanied by the covalent bonding of a cholesterol moiety to the C terminus of this N-terminal portion, producing the active Hh. The unmodified Hh protein exerts a broader range of Hh action than is normally observed (Porter et al., Cell 86:21-24, 1996). The present invention describes a new class of proteins, Dispatched, which modulate lipid modified proteins e.g. Hedgehog.

### BRIEF SUMMARY OF THE INVENTION

Hence, it is a general object of the invention to provide a class of novel sterol-sensing domain proteins called “dispatched” which modulate lipid modified hydrophobic proteins and nucleic acids coding for respective proteins. Such proteins e.g. are involved in developmental processes, differentiation, regeneration and homeostasis, making them particularly suitable tools for diagnosis and therapy.

Another object of the present invention are dispatched (disp) proteins of said family having the sequence as specified in the Table 5 and SEQ. ID. NO 1 and homologues of said sequence comprising proteins which contain a contiguous stretch of amino acids that fulfills the following conditions in a blastp comparison to Drosophila dispatched (dmDispatched):

TABLE 1
Blastp comparison

| Length of contiguous | Minimal percentage |
|---|---|
| stretch of amino acids | of identities |
| 100 aa | 35 |
| 150 aa | 30 |
| 200 aa | 27 |

Another object of the present invention are dispatched proteins of said family having the sequence as specified in Table 5 and SEQ. ID. NO 1 and homologues of said sequence comprising proteins whose full length amino acid sequence shows at least one of the following percentages of identities, when it is aligned by Clustal W to the indicated parts of dmDispatched.

TABLE 2
Clustal W comparison

| Part of dmDispatched used in | Minimal percentages |
|---|---|
| Clustal W (in amino acids) | of identities (in %) |
| 1-1218 (full length) | 13 |
| 442-635 (SSD domain) | 25 |
| 718-1218 | 25 |

For instance, the dispatched protein or a fragment thereof can be provided as a recombinant fusion protein which includes a second polypeptide portion e.g. a second polypeptide having an amino sequence unrelated to dispatched, this second portion can be e.g. glutathione-S-transferase, alkaline phosphatase or an epitope tag.

Another aspect of the present invention provides antibodies and antibody preparations specifically reactive with an epitope of the dispatched protein.

Another object of the present invention is a nucleotide sequence which encodes a dispatched protein. The coding sequence of the nucleotide sequence comprises all sequences encoding the amino acid sequence of SEQ. ID. NO. 1 or homologues thereof or partial sequences thereof as described above, such as a nucleotide sequence comprising a nucleotide sequence which is identical to the coding sequence represented in the SEQ ID NO: 1. For instance, the dispatched encoding sequence preferably has a sequence at least 20% homologous to the nucleotide sequence encoding the amino acid sequence set forth in the Table 5 and in SEQ. ID. NO. 1, preferably a sequence that is at least 30 % homologous to the nucleotide sequence of SEQ ID NO: 1, though higher sequence homologies of, for example, 40%, 50% or 60% are also contemplated.

Another object of the present invention is a method for the secretion of lipid modified proteins comprising a dispatched expression system which includes at least one transcriptional promoter or transcriptional enhancer sequence operably linked to the dispatched nucleotide sequence, in a suitable host cell capable of hydrophobic e.g. hedgehog protein expression and isolation of the secreted proteins from the medium.

Another object of the present invention are non human transgenic animals having a transgene, e.g. animals which express a mutated or non mutated heterologous sequence of a dispatched gene, e.g. incorporated into their genome or animals which have at least one of their endogenous dispatched genes disrupted.

Another object of the present invention is a screening method for agonists or antagonists of dispatched protein activity. An exemplary method includes the expression of a dispatched protein in a host cell capable of expressing a hydrophobic or hydrophobised protein, such as hedgehog protein, treating this cell with a test compound and measuring the content of free hedgehog protein and membrane bound hedgehog protein. A statistically significant shift in favor of the free hedgehog protein is indicative for a test compound with agonist activity.

Another object of the present invention is a method of inducing and/or maintaining a differentiated state, causing proliferation, and/or enhancing survival of a cell responsive to a hedgehog protein, by contacting the cells with a dispatched agonist.

Another object of the present invention are dispatched proteins of said family which are characterized by a changed dispatched activity for example an upregulation or downregulation of activity, in particular members of said family having the sequence as specified in SEQ. ID. NO 1 or homologues of said sequence, in particular the homologues with the best matching protein sequences determined by a blastp comparison or a Clustal W comparison with program parameters as defined herein.

Another object of the present invention is a method of determining whether a subject, e.g. a human patient, is at risk for a disorder characterized by unwanted cell proliferation or aberrant control of differentiation. The method includes detecting, in a tissue sample of the subject, the presence or absence of a genetic lesion characterized by at least one mutation in a dispatched gene or the misexpression of a dispatched gene.

Another object of the present invention is the use of isolated nucleic acid in antisense therapy comprising the administration or in situ generation of oligonucleotide probes or their derivatives which specifically hybridizes under cellular conditions with the cellular mRNA and/or genomic DNA encoding a dispatched protein so as to inhibit expression of said protein.

Other features and advantages of the invention will become apparent from the following detailed description and from the claims.

The invention will be better understood and objects other than those set forth above will become apparent when consideration is given to the following detailed description.

### DETAILED DESCRIPTION OF THE INVENTION

Hence, it is a general object of the invention to provide a class of novel sterol-sensing domain proteins, called dispatched which modulate lipid modified hydrophobic proteins and nucleic acids coding for respective proteins. Such proteins e.g. are involved in developmental processes, differentiation, regeneration and cell homeostasis, making them particularly suitable tools for diagnosis and therapy.

As used herein the term hydrophilizing refers to an activity comprising the release of Hedgehog proteins and/or lipid modified proteins from cells e.g. releasing hedgehog proteins from cells. As used herein the term dispatched refers to sterol sensing domain proteins which modulate lipid modified hydrophobic proteins and comprises homologuous proteins.

The present invention makes available for the first time members of the novel family of so called dispatched proteins. Preferably the dispatched proteins as defined by their structural homology or identity to the sequence of the Table 5 or SEQ ID NO: 1, wherein homology comprises proteins which contain a contiguous stretch of amino acids that fulfills the following conditions in a blastp comparison to dmDispatched:

TABLE 3
Blastp comparison

| Length of contiguous | Minimal percentage |
|---|---|
| stretch of amino acids | of identities |
| 100 aa | 35 |
| 150 aa | 30 |
| 200 aa | 27 |

The Parameters of the blastp program were the following:

Program: blastp (aa sequence against aa sequence)

Matix: blosum 62

Gap open penalty: 11

Gap extension penalty: 1

X_dropoff: 50

Expect: 10.0

Wordsize: 3

Filter: none

Subject: dmDisp

Query: protein of interest

Reference: Tatiana A. Tatusova, Thomas L. Madden (1999), “Blast 2 sequences—a new tool for comparing protein and nucleotide sequences”, FEMS Microbiol Lett. 174:247-250.

The present invention makes available for the first time members of the novel family of so called dispatched proteins. Preferably the dispatched proteins as defined by their structural homology or identity to the sequence of the Table 5 or SEQ ID NO:1, wherein homology comprises proteins whose full length amino acid sequence shows at least one of the following percentages of identities, when it is aligned by Clustal W to the indicated parts of dmDispatched.

TABLE 4
Clustal W comparison

| Part of dmDispatched used | Minimal percentages |
|---|---|
| in Clustal W (in amino acids) | of identities |
| 1-1218 (full length) | 13 |
| 442-635 (SSD domain) | 25 |
| 718-1218 | 25 |

The Parameters of the Clustal W program were the following:

pairwise alignment

matrix: blosum 30

open gap penalty: 80.0

extend gap penalty: 0.01

multiple alignment

matrix: blosum series

open gap penalty: 80.0

extend gap penalty: 0.01

delay divergent: .40%

Reference:

Thompson, J. D., Higgins, D. G., Gibson T. J. (1994), CLUSTAL W: improving the sensitivity of progressive multiple sequence alignment through sequence weighting, position-specific gap penalties and weight matrix choice. Nucleic Acids Res. 22(22): 4673-80. As examples for sequences fulfilling the above defined conditions see SEQ ID NO:3 and SEQ ID NO:4.

Included within the term dispatched are also functional fragments, variants or derivatives of any of the proteins defined hereinbefore. The proteins of the present invention can be provided as chimeric proteins for example as recombinant fusion proteins.

A “chimeric protein” or “fusion protein” is a fusion of a first amino acid sequence encoding one of the subject dispatched polypeptides with a second amino acid sequence defining a domain foreign to and not substantially homologuous with any domain of one of the vertebrate hh proteins. A chimeric protein may present a foreign domain which is found (albeit in a different protein) in an organism which also expresses the first protein, or it may be an “interspecies”, “intergenic”, etc. fusion of protein structures expressed by different kinds of organisms. In general, a fusion protein can be represented by the general formula X-disp-Y, wherein disp represents a portion of the protein which is derived from one of the vertebrate dispatched proteins, and X and Y are independently absent or represent amino acid sequences which are not related to one of the vertebrate dispatched sequences in an organism, including naturally occurring mutants.

Another object of the present invention is a nucleotide sequence which encodes a dispatched protein. The coding sequence of the nucleotide sequence of the present invention comprises all sequences encoding the amino acid sequence of the Table 5 and SEQ. ID. NO. 1 or homologuous sequences thereof or partial sequences thereof as described above, preferably a sequence which is identical to the coding sequence represented in the SEQ ID NO:1. For instance, the dispatched encoding sequence preferably has a sequence at least 20% homologuous to the nucleotide sequence encoding the amino acid sequence set forth in SEQ. ID. NO. 1, preferably at least 30 % homologuous to the nucleotide sequence of SEQ ID NO: 1, though higher sequence homologies of, for example, 40%, 50% or 60% are also contemplated. It has to be understood that the nucleotide sequences of the present invention—beside the dispatched encoding sequence—can comprise further sequences known to skilled person as necessary or favorably to express the respective sequence alone or together with further sequences. Wherever nucleotide sequences are at issue in the scope of the present invention, the complementary strands of specified sequences are of course also comprised.

In one embodiment, the nucleic acid is a cDNA encoding a peptide having at least one activity of the subject Drosophila polypeptide.

DNA sequence polymorphisms that do lead to changes in the amino acid sequence of the subject dispatched proteins are also comprised by the present invention. One skilled in the art will appreciate that these variations in one or more nucleotides (up to about 3-5% of the nucleotides) of the nucleic acids encoding polypeptides having an activity of a dispatched polypeptide may exist among individuals of a given species due to natural allelic variation. Fragments of the nucleic acids encoding an active portion of the dispatched proteins are also within the scope of the invention. As used herein, a dispatched gene fragment refers to a nucleic acid having fewer nucleotides than the nucleotide sequence encoding the entire amino acid sequence of the dispatched protein represented in SEQ ID No. 1, yet preferably encodes a peptide which retains some biological activity of the full length protein or regains some biological activity in the presence of a suitable agonist/antagonist. Nucleic acid fragments within the scope of the present invention include those capable of hybridizing under high or medium stringency conditions with nucleic acids from other species for use in screening protocols to detect and isolate other dispatched alleles and/or homologs, as well as those capable of hybridizing with nucleic acids from human specimens for use in detecting the presence of a nucleic acid encoding a dispatched protein, including alternate isoforms, e.g. mRNA splicing variants. Nucleic acids within the scope of the invention may also contain linker sequences, modified restriction endonuclease sites and other sequences useful for molecular cloning, expression or purification of recombinant forms of the subject dispatched polypeptides.

Another object of the present invention is a method for the production of hydrophobized e.g. lipid modified proteins which method comprises a dispatched expression system which includes at least one transcriptional promoter and/or transcriptional enhancer sequence operably linked to the dispatched nucleotide sequence, in a suitable host cell capable of hedgehog expression and isolation of the secreted proteins from the medium. This is based on the findings that e.g. Drosophila dispatched protein (See Table 5 and SEQ ID NO:1) is involved in the release of lipid modified Hh protein from Hh producing cells.

In an illustrative embodiment, a host cell transfected with a nucleic acid vector directing expression of a nucleotide sequence encoding a dispatched protein can be cultured under appropriate conditions to allow expression of the peptide to occur. In a preferred embodiment the host cell is a eucaryotic cell and in a more preferred embodiment the host cell is a mammalian cell. The hydrophobized polypeptide e.g. hedgehog is in the presence of dispatched secreted and can be isolated from a mixture of cells and medium containing the recombinant or endogenous polypeptide. The secreted polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. A cell culture includes host cells, media and other byproducts. Suitable media for cell culture are well known in the art.

In a preferred embodiment, the polypeptide is recombinant hedgehog polypeptide and in a more preferred embodiment the hh polypeptide is a fusion protein containing a domain which facilitates its purification, such as an hedgehog/GST fusion protein.

Another aspect of the invention relates to the use of the isolated nucleic acid in “antisense” therapy. As used herein, “antisense” therapy refers to administration or in situ generation of oligonucleotide probes or their derivatives which specifically hybridizes (e.g. binds) under cellular conditions, with the cellular mRNA and/or genomic DNA encoding one or more of the dispatched proteins so as to inhibit expression of that protein, e.g. by inhibiting transcription and/or translation. The binding may be by conventional base pair complementarity, or, for example, in the case of binding to DNA duplexes, through specific interactions in the major groove of the double helix. In general, “antisense” therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular mRNA which encodes a dispatched protein. Alternatively, the antisense construct is an oligonucleotide probe which is generated ex vivo and which, when introduced into the cell causes inhibition of expression by hybridizing with the mRNA and/or genomic sequences of a dispatched gene. Such oligonucleotide probes are preferably modified oligonucleotide which are resistant to endogenous nucleases, e.g. exonucleases and/or endonucleases, and is therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) Biotechniques 6:958-976; and Stein et al. (1988) Cancer Res 48:2659-2668.

Accordingly, the modified oligomers of the invention are useful in therapeutic, diagnostic, and research contexts. In therapeutic applications, the oligomers are utilized in a manner appropriate for antisense therapy in general. For such therapy, the oligomers of the invention can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intra-peritoneal, and subcutaneous for injection, the oligomers of the invention can be formulated in liquid solutions, preferably in physiologically compatible buffers such as Hank's solution or Ringer's solution. In addition, the oligomers may be formulated in solid form and redissolved or suspended immediately prior to use. Lyophilized forms are also included.

Systemic administration can also be by transmucosal or transdermal means, or the compounds can be administered orally. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For oral administration, the oligomers are formulated into conventional oral administration forms such as capsules, tablets, and tonics. For topical administration, the oligomers of the invention are formulated into ointments, salves, gels, or creams as generally known in the art.

In addition to use in therapy, the oligomers of the invention may be used as diagnostic reagents to detect the presence or absence of the target DNA or RNA sequences to which they specifically bind. Such diagnostic tests are described in further detail below. Likewise, the antisense constructs of the present invention, by antagonizing the normal biological activity of one of the dispatched proteins, can be used in the manipulation of tissue, e.g. tissue differentiation, both in vivo and in ex vivo tissue cultures.

Also, the anti-sense techniques (e.g. micro-injection of antisense molecules, or transfection with plasmids whose transcripts are anti-sense with regard to an dispatched mRNA or gene sequence) can be used to investigate role of dispatched in developmental events, as well as the normal cellular function of dispatched in adult tissue. Such techniques can be utilized in cell culture, but can also be used in the creation of transgenic animals.

Another object of the present invention is a method of determining whether a subject, e.g. a human patient, is at risk for a disorder characterized by unwanted cell proliferation or aberrant control of differentiation. The method includes detecting, in a tissue sample of the subject, the presence or absence of a genetic lesion characterized by at least one mutation in a dispatched gene or the mis-expression of a dispatched gene.

To illustrate, nucleotide probes can be generated from the subject dispatched genes which facilitate histological screening of intact tissue and tissue samples for the presence (or absence) of dispatched-encoding transcripts. The use of probes directed to dispatched messages, or to genomic dispatched sequences, can be used for both predictive and therapeutic evaluation of allelic mutations which might be manifest in, for example, neoplastic or hyperplastic disorders (e.g. unwanted cell growth) or abnormal differentiation of tissue. The oligonucleotide probes can help facilitate the determination of the molecular basis for a developmental disorder which may involve some abnormality associated with expression (or lack thereof) of a dispatched protein. For instance, variation in polypeptide synthesis can be differentiated from a mutation in a coding sequence.

In preferred embodiments, the subject method can be gene rally characterized as comprising: detecting, in a tissue sample of the subject (e.g. a human patient), the presence or absence of a genetic lesion characterized by at least one of (i) a mutation of a gene encoding a dispatched protein or (ii) the mis-expression of a dispatched gene. To illustrate, such genetic lesions can be detected by ascertaining the existence of at least one of (i) a deletion of one or more nucleotides from a dispatched gene, (ii) an addition of one or more nucleotides to a dispatched gene, (iii) a substitution of one or more nucleotides of a dispatched gene, (iv) a gross chromosomal rearrangement of a dispatched gene, (v) a gross alteration in the level of a messenger RNA transcript of an dispatched gene, (vi) the presence of a non-wild type splicing pattern of a messenger RNA transcript of a vertebrate dispatched gene, and (vii) a non-wild type level of a dispatched protein. In one aspect of the invention there is provided a probe/primer comprising an oligonucleotide containing a region of nucleotide sequence which is capable of hybridizing to a sense or antisense sequence of SEQ ID No:1, or naturally occurring mutants thereof, or 5′ or 3′ flanking sequences or intronic sequences naturally associated with a vertebrate dispatched gene. The probe is exposed to nucleic acid of a tissue sample; and the hybridization of the probe to the sample nucleic acid is detected. In certain embodiments, detection of the lesion comprises utilizing the probe/primer in a polymerase chain reaction (PCR) (see, e.g., U.S. Pat. No. 4,683,195 and 4,683,202) or, alternatively, in a ligation chain reaction (LCR) (see, e.g., Landegran et al. (1988) Science, 241:1077-1080; and NaKazawa et al. (1944) PNAS 91:360-364) the later of which can be particularly useful for detecting point mutations in dispatched genes. Alternatively, immunoassays can be employed to determine the level of dispatched proteins.

Another aspect of the present invention provides antibodies and antibody preparations specifically reactive with an epitope of-the dispatched protein.

For example, by using immunogens derived from dispatched proteins, e.g. based on the cDNA sequences, anti-protein/anti-peptide antisera or monoclonal antibodies can be made by standard protocols (See, for example, Antibodies: A Laboratory Manual ed. by Harlow and Lane (Cold Spring Harbor Press: 1988)). A mammal, such as a mouse, a hamster or rabbit can be immunized with an immunogenic form of the peptide (e.g., a vertebrate dispatched polypeptide or an antigenic fragment which is capable of eliciting an antibody response). Techniques for conferring immunogenicity on a protein or peptide include conjugation to carriers or other techniques well known in the art. An immunogenic portion of a dispatched protein can be administered in the presence of adjuvant. The progress of immunization can be monitored by detection of antibody titers in plasma or serum. Standard ELISA or other immunoassays can be used with the immunogen as antigen to assess the levels of antibodies.

In a preferred embodiment, the subject antibodies are immunospecific for antigenic determinants of a dispatched protein of a vertebrate organism, such as a mammal. Following immunization of an animal with an antigenic preparation of a dispatched protein, anti-dispatched antisera can be obtained and, if desired, polyclonal anti-dispatched antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al.,(1985) Monoclonal Antibodies and Cancer Therapy, Alan R. Liss, Inc. pp. 77-96). Hybridoma cells can be screened immunochemically for production of antibodies specifically reactive with a disp polypeptide of the present invention and monoclonal antibodies isolated from a culture comprising such hybridoma cells.

The term antibody as used herein is intended to include fragments thereof which are also specifically reactive with one of the subject dispatched polypeptides. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific and chimeric molecules having affinity for a dispatched protein conferred by at least one CDR region of the antibody.

Both monoclonal and polyclonal antibodies (Ab) directed against authentic dispatched polypeptides, or dispatched variants, and antibody fragments such as Fab and F(ab)₂, can be used to block the action of one or more dispatched proteins and allow the study of the role of these proteins in, for example, embryogenesis and/or maintenance of differential tissue. In a similar approach, hybridomas producing anti-dispatched monoclonal antibodies, or biodegradable gels in which anti-dispatched antibodies are suspended, can be implanted at a site proximal or within the area at which dispatched action is intended to be blocked. Experiments of this nature can aid in deciphering the role of this and other factors that may be involved in limb patterning and tissue formation.

Another aspect of the present invention concerns transgenic animals which are comprised of cells (of that animal) which contain a transgene of the present invention and which preferably (though optionally) express an exogenous dispatched protein in one or more cells in the animal. A dispatched transgene can encode the wild-type form of the protein, or can encode homologues thereof, including both agonists and antagonists, as well as antisense constructs. In preferred embodiments, the expression of the transgene is restricted to specific subsets of cells, tissues or developmental stages utilizing, for example, cis-acting sequences that control expression in the desired pattern. In the present invention, such mosaic expression of a dispatched protein can be essential for many forms of lineage analysis and can additionally provide a means to assess the effects of, for example, lack of dispatched expression which might grossly alter development in small patches of tissue within-an otherwise normal embryo. Toward this tissue-specific regulatory sequences and conditional regulatory sequences can be used to control expression of the transgene in certain spatial patterns. Moreover, temporal patterns of expression can be provided by, for example, conditional recombination systems or prokaryotic transcriptional regulatory sequences. Genetic techniques which allow for the expression of transgenes can be regulated via site-specific genetic manipulation in vivo. Such techniques are known to those skilled in the art.

For instance, genetic systems are available which allow for the regulated expression of a recombinase that catalyzes the genetic recombination of a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity. Recombinase catalyzed recombination events can be designed such that recombination of the target sequence results in either the activation or repression of expression of one of the subject dispatched proteins.

In an illustrative embodiment, either the cre/loxP recombinase system of bacteriophage P1 (Lakso et al. (1992) PNAS 89:6232-6236; Orban et al. (1992) PNAS 89:6861-6865) or the FLP recombinase system of Saccharomyces cerevisiae (O'Gorman et al. (1991) Science 251:1351-1355; PCT publication WO 92/15694) can be used to generate in vivo site-specific genetic recombination systems. Cre recombinase catalyzes the site-specific recombination of an intervening target sequence located between loxP sequences. LoxP sequences are 34 base pair nucleotide repeats sequences to which the Cre recombinase binds and are required for Cre recombinase mediated genetic recombination. The orientation of loxp sequences determines whether the intervening target sequence is excised or inverted when Cre recombinase is present (Abremski et al. (1984) J Biol. Chem. 259:1509-1514); catalyzing the excision of the target sequence when the loxP sequences are oriented as direct repeats and catalyzes inversion of the target sequence when loxP sequences are oriented as inverted repeats. Accordingly, genetic recombination of the target sequence is dependent on ex-pression of the Cre recombinase. Expression of the recombinase can be regulated by promotor elements which are subject to regulatory control, e.g., tissue-specific, developmental stage-specific, inducible or repressible by externally added agents. This regulated control will result in genetic recombination of the target sequence only in cells where recombinase expression is mediated by the promotor element. Thus, the activation expression of a recombinant dispatched protein can be regulated via control of recombinase expression.

Use of the cre/loxP recombinase system to regulate expression of a recombinant dispatched protein requires the construction of a transgenic animal containing transgenes encoding both the Cre recombinase and the subject protein. Animals containing both the Cre recombinase and a recombinant dispatched gene can be provided through the construction of “double” transgenic animals. A convenient method for providing such animals is to mate two transgenic animals each containing a transgene, e.g., a dispatched gene and recombinase gene. One advantage derived from initially constructing transgenic animals containing a dispatched transgene in a recombinase-mediated expressible format derives from the likelihood that the subject protein, whether agonistic or antagonistic, can be deleterious upon expression in the transgenic animal. In such an instance, a founder population, in which the subject transgene is silent in all tissues, can be propagated and maintained. Individuals of this founder population can be crossed with animals expressing the recombinase in, for example, one or more tissues and/or a desired temporal pattern. Thus, the creation of a founder population in which, for example, an anta-gonistic dispatched transgene is silent will allow the study of progeny from that founder in which disruption of dispatched mediated induction in a particular tissue or at certain developmental stages would. result in, for example, a lethal phenotype. Similar conditional transgenes can be provided using prokaryotic promotor sequences which require prokaryotic proteins to be simultaneously expressed in order to facilitate expression of the dispatched transgene. Exemplary promoters and the corresponding trans-activating prokaryotic proteins are given in U.S. Pat. No. 4,833,080.

Moreover, expression of the conditional transgenes can be induced by gene therapy-like methods wherein a gene encoding the trans-activating protein, e.g. a recombinase or a prokaryotic protein, is delivered to the tissue and caused to be expressed, such as in a cell-type specific manner. By this method, a dispatched transgene could remain silent into adulthood until “turned on” by the introduction of the trans-activator.

In an exemplary embodiment, the “transgenic non-human animals” of the invention are produced by introducing transgenes into the germline of the non-human animal.

Embryonic target cells at various developmental stages can be used to introduce transgenes. Different methods are used depending on the stage of development of the embryonic target cell. The zygote is the best target for micro-injection. In the mouse, the male pronucleus reaches the size of approximately 20 micrometers in diameter which allows reproducible injection of 1-2 pl of DNA solution. The use of zygotes as a target for gene transfer has a major advantage in that in most cases the injected DNA will be incorporated into the host gene before the first cleavage (Brinster et al. (1985) PNAS 82:4438-4442). As a consequence, all cells of the transgenic non-human animal will carry the incorporated transgene. This will in general also be reflected in the efficient transmission of the transgene to offspring of the founder since 50% of the germ cells will harbor the transgene. Microinjection of zygotes is the preferred method for incorporating transgenes in practicing the invention.

Retroviral infection can also be used to introduce dispatched transgenes into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

A third type of target cell for transgene introduction is the embryonic stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

Methods of making dispatched knock-out or disruption transgenic animals are also generally known. See, for example, Manipulating the Mouse Embryo, (Cold Spring Harbor Laboratory Press, Cold Spring Harbor, N. Y., 1986) Recombinase dependent knockouts can also be generated, e.g. by homologous recombination to insert recombinase target sequences flanking portions of an endogenous dispatched gene, such that tissue specific and/or temporal control of inactivation of a dispatched allele can be controlled as above.

Another object of the present invention is a screening method for agonists or antagonists of dispatched protein activity. Due to the changes observable in embryonic, larval and adult stages, in particular Drosophila are very suitable in vivo screening systems for both agonists and antagonists to dispatched. Such flies can either be wild-type flies (for antagonist search)or dispatched mutant flies (for agonist search) or they can comprise a dispatched mutant with enhanced or reduced activity for both agonist and antagonist screening. In a preferred embodiment of said object, a fly e.g. a dispatched mutant fly, in particular a dispatched mutant Drosophila melanogaster organism is used in a screening assay comprising contacting eggs of said fly with a test compound and analysing the resulting phenotypes. In an as well preferred embodiment the method includes the expression of a dispatched protein in a host cell capable of expressing hedgehog protein, contacting this cell with a test compound e.g. peptide or non-peptide agents and for example measuring the content of free hedgehog protein and membrane bound hedgehog protein. A statistically significant shift in favor of the free hedgehog protein is indicative for a test compound with dispatched agonist activity. In a more preferred embodiment the host cell is a eucaryotic cell and in a even more preferred embodiment the cell is a mammalian cell.

In a further embodiment the present invention provides a method for identifying compounds capable of binding to dispatched. Said method comprises a protein of the invention in a binding assay allowing the identification of synthetic or natural dispatched binding partners. More specifically said binding assay involves exposure of a protein of the invention to a test compound under conditions sufficient to allow binding of said test compound to said protein of the invention and determining qualitatively and/or quantitatively whether binding has occurred e.g. . by detecting the complex formed between the test compound and the dispatched protein. Binding of the test compound to the protein of the invention can be detected by methods well known in the art.

Another object of the present invention is a method of inducing and/or maintaining a differentiated state, causing proliferation, and/or enhancing survival of a cell responsive to a hedgehog protein, by contacting the cells with a dispatched agonist.

For instance, it is contemplated by the invention that, in light of the finding of an apparently broad involvement of hedgehog proteins in the formation of ordered spatial arrangements of differentiated tissues in vertebrates and the finding of the present invention that dispatched functions in hedgehog signaling, the subject method could be used to generate and/or maintain an array of different vertebrate tissue both in vitro and in vivo. A dispatched agent, whether inductive or anti-inductive, can be, as appropriate, any of the preparations described above, including gene therapy constructs, antisense molecules, peptidomimetics or agents identified in the screening assays provided herein.

Many neurological disorders are associated with degeneration of discrete populations of neuronal elements and may be treatable with a therapeutic regimen which includes a dispatched agonist. For example, Alzheimer's disease is associated with deficits in several neurotransmitter systems, both those that project to the neocortex and those that reside with the cortex. For instance, the nucleus basalis in patients with Alzheimer's disease have been observed to have a profound (75%) loss of neurons compared to age-matched controls. Although Alzheimer's disease is by far the most common form of dementia, several other disorders can produce dementia. Several of these are degenerative diseases characterized by the death of neurons in various parts of the central nervous system, especially the cerebral cortex. However, some forms of dementia are associated with degeneration of the thalamus or the white matter underlying the cerebral cortex. Here, the cognitive dysfunction results from the isolation of cortical areas by the degeneration of efferent and affronts. Huntington's disease involves the degeneration of intrastraital and cortical cholinergic neurons and GABAergic neurons. Pick's disease is a severe neuronal degeneration in the neocortex of the frontal and anterior temporal lobes, sometimes accompanied by death of neurons in the striatum. Treatment of patients suffering from such degenerative conditions can include the application of agents which mimic the effects of dispatched proteins, in order to control, for example, differentiation and apoptotic events which give rise to loss of neurons (e.g. to enhance survival of existing neurons) as well as promote differentiation and repopulation by progenitor cells in the area affected. In preferred embodiments, a source of a dispatched agent is stereotactically provided within or proximate the area of degeneration. In addition to degenerative-induced dementias, a pharmaceutical preparation of one or more of the dispatched agent can be applied opportunely in the treatment of neuro-degenerative disorders which have manifestations of tremors and involuntary movements. Parkinson's disease, for example, primarily affects subcortical structures and is characterized by degeneration of the nigrostriatal pathway, raphe nuclei, locus ceruleus, and the motor nucleus of vagus. Ballism is typically associated with damage to the subthalamic nucleus, often due to acute vascular accident. Also included are neurogenic and myopathic diseases which ultimately affect the somatic division of the peripheral nervous system and are manifest as neuromuscular disorders. Examples include chronic atrophies such as amyotrophic lateral sclerosis, Guillain-Barre syndrome and chronic peripheral neuropathy, as well as other diseases which can be manifest as progressive bulbar palsies or spinal muscular atrophies. The present method is amenable to the treatment of disorders of the cerebellum which result in hypotonia or ataxia, such as those lesions in the cerebellum which produce disorders in the limbs ipsilateral to the lesion. For instance, a preparation of a dispatched agent can be used to treat a restricted form of cerebellar cortical degeneration involving the anterior lobes (vermis and leg areas) such as is common in alcoholic patients.

In an illustrative embodiment, the subject method is used to treat amyotrophic lateral sclerosis. ALS is a name given to a complex of disorders that comprise upper and lower motor neurons. Patients may present with progressive spinal muscular atrophy, progressive bulbar palsy, primary lateral sclerosis, or a combination of these conditions. The major pathological abnormality is characterized by a selective and progressive degeneration of the lower motor neurons in the spinal cord and the upper motor neurons in the cerebral cortex. The therapeutic application of a dispatched agonist can be used alone, or in conjunction with other neurotrophic factors such as CNTF, BDNF or NGF to prevent and/or reverse motor neuron degeneration in ALS patients.

Furthermore, a potential role for certain of the hedgehog proteins in development and maintenance of dendritic processes of axonal neurons and the functioning of dispatched in the hedgehog signaling pathway make dispatched agents potential candidates which can be employed to support, or alternatively antagonize the survival and reprojection of several types of ganglionic neurons sympathetic and sensory neurons as well as motor neurons. In particular, such therapeutic compositions may be useful in treatments designed to rescue, for example, various neurons from lesion-induced death as well as guiding reprojection of these neurons after such damage. Such diseases include, but are not limited to, CNS trauma infarction, infection (such as viral infection with varicella-zoster), metabolic disease, nutritional deficiency, toxic agents (such as cisplatin treatment). Moreover, certain of the dispatched agents (such as antagonistic form) may be useful in the selective ablation of sensory neurons, for example, in the treatment of chronic pain syndromes.

In the following Examples the isolation and characterization of the Drosophila dispatched gene is described. It has, however, to be understood that the same procedure is also applicable to other organisms with adaptations obvious to the skilled person.

### Identification of Dispatched, a Novel Gene Required for Hh Signaling

In a genetic screen for new components of the Drosophila Hh signaling pathway, we identified a mutation on the third chromosome causing phenotypes typical of those resulting from loss of hh or wg function. Animals zygotically homozygous for this mutation survive until early pupal stages. However, animals lacking in addition the maternal component of this locus die during embryogenesis with a strong segment-polarity phenotype. Instead of the wild-type segmentally repeated pattern of denticle belts interspersed by naked such embryos display a lawn of denticle belts and fail to secrete naked ventral cuticle. Germ line clone-derived embryos are rescued by a wild-type paternal chromosome, indicating that the gene product is required only after the onset of zygotic transcription. Segment-polarity phenotypes are indicative of loss-of-function mutations in essential components of the Hh and Wg signal transduction pathways (Nüsslein-Volhard and Wieschaus, 1980). Due to its presumed role and the structural similarities and functional dissimilarities to ptc described below, we have named this new gene dispatched (disp).

To determine if disp is required specifically for either the Hh or Wg pathway, we generated large disp^{-/-} clones in the adult wing, a tissue in which the two pathways function independently of each other in distinct sub-populations of cells. Loss of Wg signaling in the wing primordium results in loss of wing margin (Couso et al., 1994), whereas a reduction in Hh activity causes a strong narrowing of the intervein region between longitudinal veins L3 and L4 (Slusarski et al., 1995; Sanchez-Herrero et al., 1996; Methot and Basler, 1999). Although disp^{-/-} clones can encompass large regions of Wg-sending and Wg-receiving cells, they contribute to wild-type wing margin structures, which indicates that disp function is not required for the Wg signaling pathway. However, when located in the posterior compartment, large clones cause a significant reduction in the distance between veins L3 and L4, a phenotype typical for the reduction of Hh signaling at the A/P. Thus, we conclude that disp is acting in the Hh signaling pathway.

### Disp Encodes a Putative Multi-pass Transmembrane Protein With a Sterol-sensing Domain

We mapped the disp gene to cytological position 83C, cloned genomic sequences of disp, and isolated corresponding cDNA clones (see Experimental Procedures). The composite sequence from these cDNAs revealed an open reading frame (ORF) encoding a putative protein of 1218 amino acids (See table 5).

TABLE 5

Amino acid sequence of D. melanogaster
dispatched in single letter code

MLCFDSERMNWYYHVLARRPYLVVVSIAVYCVACIIVA
LVLNKLPDFSDPTLGFETRGTKIGERLTAWYNLLQETDHHGALFSNPSDL
WERRRVEQGYVETKLHPNHRRRKNKHKNRNKNKRRKEQNQSSHEHHDVAQ
KMMQFKKRLKATSSPSPNLGFDTWIGDSGVFRDYEITNDSASSSLEPTRR
TEQIEYGHNTTSVDEEEHQQRVQTKKSTWRLLKQAATLPTDGWADMHRRQ
PIEGFFCDSSPRKEYSHFVVQRIGPNATDSLFDLNGLLAMCQLQDQITEV
PSYRAFCEPEMLTTECCRPWSLPNYAAMLANKSSCFDLTTEDVTSLHTLL
LGCYEYFHDLKMDNHCNEIPHCRAPEECKRLNIVFNVLNFLTDFSFIKSN
DSNVYLKYAMIFIPVAQSNRLLPLFHEWEDVELINELVEVVAMDLGLENE
LFNELLLTDVWLVSLGGTFVMASVWLYTGSAFITLMSCVAICFSLGLAYF
FYAIVLEFEFFPYMNLLAVVVIIGIGADDVFLFLKIWHCVLTERFSNRCT
LTTQSQSALPTLENSDHTESLENIMALTMRHAAASMFVTSLTTAGAFYAS
YSSSITAIKCFGIFAGTVVVTNYLLMITWLPASVSIMERLFATRMSCHHP
MSIKLIHACKKSINRFCQMFEECITKSIMNYAYLWLLIFGALGASSAVIV
FWYPGLQLPEKSHFQLFVSKHPFEVYSSLKQQFWFEKPWQAYENFKMHMH
FVWGVQAVDDGDYTNPNSYGHLHYDNNFNVSSRPAQLWILDFCQSVRQQP
FYKETLGMLLPNCFIENLIDYMKRRCIDDMDSTRKDRSPCCDAQFPFEPH
IFEYCLPQSISNMYDTTFFRPGVAGPKFAEAPRLETEDYLGMSGNESAEY
STNGSFTPLLVKALVIEFESNVAYSTIYANIRQFYESVEHWFQMQLKTAP
PELQGGWFTSDLKFYNVQDTLSHDTFVAICLAMAASLAVLLCFTVNILIS
IYAVLTVSLSIFNTVAVLILLGWQLNILESIAVSTAIGLAVDFSLHYGIH
YRMSPVKERLAATQFVLSRIIGPTVMAATTTGLAGGIMMASNILPYIQIG
VFLVVVMIVSWFYATFFLMSLLRVAGPQHGFLELKWPLWSKRSSGSSKFY
ERKPSQVIASEQLLTPTSSAIVELANSETHELESLNSNSLIKTISGIESA
HALSSLPRDFEHSFQTMHECKYQTYPSTSN 1218

Underlined is the sterol sensing domain (SSD).

A transgene containing the full-length ORF driven by the weak, ubiquitous promoter of the tubulinα1 gene was introduced into the Drosophila germline and fully rescued disp^{-/-} animals to viable adults, confirming that the cloned gene is indeed responsible for the pupal lethality and wing phenotype caused by the disp mutation. In addition, rescued animals are fully fertile when crossed inter se, indicating that the transgene also rescues the embryonic segment-polarity phenotype associated with the absence of disp function.

Searches of genome data bases revealed structural homologies of the Disp protein to the products of the vertebrate ptc (Goodrich et al., 1996; Marigo et al., 1996; Hahn et al., 1996) and NPC1 (Carstea et al., 1997; Loftus et al., 1997) disease genes and their Drosophila homologs (Hooper and Scott, 1989; Nakano et al., 1989). Based on the TopPred 2 (von Heijne, 1992) transmembrane domain prediction algorithm, Disp contains 12 putative membrane spanning domains. Like the Ptc and NPCl proteins, Disp has a sterolsensing domain, a domain first defined in HMG CoA reductase (Gil et al., 1985) and SCAP (Hua et al., 1996). These two proteins are key regulators of intracellular cholesterol homeostasis, while NPC1 is thought to be involved in cholesterol trafficking, since defects in this protein cause an accumulation of cholesterol in lyposomes (reviewed by Liscum and Klansek, 1998). Aside from the multi-transmembrane domain structure and the SSD, no other homologies to Ptc or NPC1 proteins could be detected in Disp. The protein with the highest overall homology to Disp is the product of an as yet uncharacterized C. elegans gene (GenBank Acc. No. AAC48001, here termed ceDisp). We propose that together, Disp and ceDisp define a new subfamily of SSD proteins.

To determine the expression pattern of disp, we probed wild-type embryos and imaginal discs with DIG-labeled sense and anti-sense disp RNA. While no staining was observed using the sense strand, ubiquitous disp expression was observed throughout the embryo and imaginal discs when the anti-sense strand was used. Thus, based on its expression pattern, disp is neither a transcriptional target nor a spatial determinant of Hh signaling.

### Disp is Required in Hh-secreting But Not Hh-receiving Cells

The adult wing clones suggested a requirement for disp function in P compartment cells. To confirm and extend this finding, we assayed the effect of disp mutant clones on Hh signaling in the wing imaginal disc, where the A/P boundary can be precisely defined, and where the transcription of the Hh target genes ptc and dpp serve as immediate readouts of Hh signaling activity. We found that even large clones of disp^{-/-} A cells abutting the A/P boundary had no discernible effect on ptc-lacZ or dpp-lacZ expression (not shown). Thus, despite its Ptc-like structure, Disp plays no role in transducing the Hh signal in responding A cells. In contrast, large clones of disp^{-/-} P cells abutting the A/P boundary caused a dramatic reduction of both ptc-lacZ and dpp-lacZ expression (not shown). As this requirement is very similar to that for hh itself, we interpret this result as evidence that Disp is essential for the effective production of Hh signal in P cells. Even a small patch of disp^{+/-} cells at the A/P boundary was sufficient to locally rescue Hh signaling, impressively demonstrating the potency of the Hh signal and its requirement for disp activity. One obvious explanation for the phenotypes associated with disp mutant cells would be an involvement of Disp in the expression of the hh gene itself. However we find that hh-lacZ expression is unaffected in disp^{-/-} clones, which rules out a requirement for Disp in hh transcription.

### Disp is a Protein Dedicated to Hh Signaling

The experiments described above show that Disp is necessary for Hh signaling in Hh producing cells. They do not address, however, whether the ubiquitously expressed Disp protein plays a role in other signaling pathways or in physiological processes. To investigate this issue, we generated animals in which disp expression was restricted to Hh-secreting cells. This was achieved by introducing a UAS-disp transgene together with a P cell-specific en-Ga14 driver into a disp mutant background. The en-Ga14 driver is inactive in A compartment cells, cells which do not secrete Hh but comprise approximately two thirds of the embryonic, larval, and adult animal. en-Ga14 is also not active in eye imaginal disc cells which do, however, secrete Hh. The en-Ga14 UAS-disp transgene combination rescued disp mutant animals to adulthood. The resulting flies displayed normal patterning in the wing, leg, notum and abdomen (not shown), and gave rise to viable offspring, which demonstrates that in larval and embryonic tissues disp function is only required in Hh producing cells. These rescued animals showed, however, a dramatic reduction in eye size (not shown), which indicates that Disp is also required for Hh signaling in the eye, and that the rescue observed in other tissues is due solely to disp⁺ transcripts provided by the en-Ga14 driver. Importantly, throughout all stages of development, A compartment cells develop and differentiate normally and become correctly patterned by numerous signaling molecules other than Hh in the complete absence of functional Disp protein. Thus, despite its ubiquitous expression, Disp is required exclusively for Hh signaling, and not for other known signaling pathways nor for sterol homeostasis or membrane integrity.

### Hh Processing Occurs Normally in Disp Mutant Cells

As disp is required in Hh-producing cells for Hh signaling, but not for hh transcription, we examined whether Disp may be required for the processing of Hh into the active signaling moiety, Hh-Np. This processing event involves the autocatalytic cleavage of full-length Hh precursor protein to the N-terminal portion Hh-N (Lee et al., 1994; Porter et al., 1995), with the concomitant covalent linkage of cholesterol to the C-terminal amino acid to form Hh-Np (Porter et al., 1996b). We assayed this cleavage event by Western blot analysis. Transgenes encoding either full-length hh cDNA (hh-F^{HA}), or only the N-terminal portion of Hh (hh-N^{HA}), were expressed under en-Ga14 control in imaginal discs. Each of these constructs was tagged with an HA epitope just N-terminal to the defined cleavage site to allow protein detection with an α-HA antibody.

In lysates of wild-type larvae expressing hh-F^{HA} two prominent bands of ˜50 and ˜30 kd were observed which are absent in lysates from control animals. These two proteins correspond to unprocessed full-length Hh and processed Hh-Np, respectively. In lysates from animals expressing tagged hh-N^{HA} only a single major protein species of ˜30 kd was detected which co-migrates with the smaller protein seen from animals expressing hh-F^{HA}, confirming that this smaller band is the result of internal cleavage of the hh-F^{HA} product. When hh-F^{HA} was expressed in disp mutant animals, the same ratio of full-length Hh to Hh-Np was observed, indicating that Hh cleavage is occurring at the same efficiency in disp mutant cells. From this result we conclude that the defect in Hh signaling imposed by the lack of Disp is not due to faulty cleavage of the Hh precursor protein. We failed to achieve conditions under which the status of cholesterol modification could be assessed. However, since the covalent addition of cholesterol is coupled to the cleavage reaction, which occurs normally in disp mutant cells, we assume that Hh-N is properly modified in the absence of Disp. In support of this assumption it should be noted that Hh lacking a C-terminal cholesterol moiety would produce an increased, rather than a decreased, spatial response to Hh. Hence we dismiss the possibility that Disp is required for cleavage and cholesterol modification of Hh.

### Retention of Hh in Disp Mutant Cells

We next investigated whether the distribution of Hh protein is altered in the absence of Disp. As observed previously by others (Tabata and Kornberg, 1994), wild-type Hh protein normally accumulates in intracellular punctuate structures in A cells near the A/P border. These accumulations of Hh antigen co-localize with punctuate Ptc staining, suggesting they might reflect vesicular signaling complexes. When disp^{-/-} discs were stained with Hh antisera, no Hh staining at all was observed in A cells, whereas staining in P cells was significantly higher than in wild-type discs. To confirm this increase in Hh levels, we generated marked disp^{-/-} clones and analyzed the distribution of Hh antigen in single discs. Strong accumulation of Hh levels in disp^{-/-} P cells was observed in comparison to neighboring wild-type P cells. Together, these results indicate that in the absence of Disp, Hh is predominantly retained in producing cells, and is thus unable to move in significant quantities to A cells. Since some weak Ptc expression is still observed in A cells of disp mutant discs, a small fraction of Hh protein must be escaping, but in vastly reduced quantities below the limits of detection.

We then asked whether the retention of Hh in disp mutant tissue might reflect defects in the intracellular trafficking of Hh protein. This possibility was raised by the observation that in embryonic epidermal cells Hh-Nu is mainly apical while Hh-Hp is predominantly basolateral, which suggests a role for the cholesterol modification in sorting (Taylor et al., 1993; Tabata and Kornberg, 1994; Porter et al., 1996a). Using Hh antisera we could not detect any specific localization along the apical/basal axis of wing imaginal disc cells, and we did not observe an alteration in Hh distribution in disp compared to wild-type. To examine whether the different isoforms might nevertheless be differently distributed in disp mutant cells, we then examined the surface distribution of Hh-F^{HA} and Hh-N^{HA} in wild-type and disp mutant tissue. In these experiments, the antibody was applied prior to fixation and permeabilization in order to visualize only cell surface antigen. In both wild-type and disp mutant tissues, Hh-F^{HA} was detected on both the basal and apical (not shown) surfaces, while Hh-N^{HA} was exclusively apical. Due to the difficulty in accurately quantifying levels of cell surface staining, we were unable to determine if the accumulation of Hh seen within disp mutant cells also occurs at the cell surface. We conclude, however, that Hh is still able to reach the surface of cells lacking Disp; and although we can not rule out that Disp is required to differentially sort some small, active fraction of total Hh protein, our results argue against a role of Disp in apical/basal sorting of Hh.

### The Cholesterol Anchor of Hh-Np is Responsible For Retention of Hh in the Absence of Disp

One candidate effector for the retention of Hh in disp mutant cells is the cholesterol moiety, which could conceivably tether Hh to the membranes of producing cells. This lipid modification has been proposed to restrict the range of Hh action, since expression of unmodified Hh-Nu results in a spatially extended Hh response in embryos (Porter et al., 1996a). Before assaying the relationship between the cholesterol modification of Hh and the function of Disp, we first further clarified the role of this modification by establishing (i) that in the absence of modification Hh-Nu possesses a vastly extended range of action in imaginal discs, (ii) that this extended range of action is an intrinsic property of Hh-Nu, and (iii) that cholesterol-free Hh-Nu is apparently not subject to sequestration by Ptc, yet retains the ability to form vesicular complexes with Ptc in receiving cells.

Expression of Hh-Nu in P cells of the wing imaginal disc results in dpp-lacZ expression in the entire A compartment of the disc, and a consequent dramatic enlargement of the A compartment. Thus Hh-Nu appears to have a range of action at least five fold larger than that of wild-type Hh. However, since this and previous experiments have been performed in the presence of endogenous Hh, it could not be ruled out that the observed extension of Hh activity depends on, or is even mediated by, endogenous Hh-Np whose range might be expanded in the presence of Hh-Nu. To address this, we created a situation where Hh-Nu is the sole source of Hh in imaginal discs by expressing hh-N^{HA} in P cells under en-Ga14 in hh^{ts2/ts2} animals that were shifted to the non-permissive temperature during early larval stages. Even in the absence of endogenous Hh, Hh-Nu is still capable of inducing the same expanded anterior compartment morphology and shows normal punctuate staining in anterior cells. Thus Hh-Nu alone is able to associate with Ptc and signal in vivo. The cholesterol anchor of Hh appears to be required for the sequestration of Hh by Ptc, since untethered Hh is seemingly unrestricted in its range. We cannot currently rule out the possibility that Hh-Nu is also, at least partially, sequestered by Ptc but that extracellular Hh-Nu levels are abnormally high and saturate the capacity of Ptc. Any sequestration of Hh-Nu must, however, be much less efficient than that of Hh-Np, since even discs containing endogenous Hh plus en-Ga14 driven Hh-Np do not show the dramatic effect caused by Hh-Nu alone.

Having confirmed that the cholesterol modification is needed for efficient Hh sequestration but not signaling, we wanted to determine if the retention of Hh in posterior disp mutant cells was due to the lipid anchor. We repeated the hh^{ts2 /ts2} hh-N^{HA} experiments in a disp mutant background so that the Hh-Nu secreting P cells were simultaneously lacking endogenous Hh and Disp. The patterning activity of cholesterol-free Hh-Nu was virtually unaffected by the lack of Disp. Hh-Nu caused the same ‘extended anterior compartment phenotype’ in hh^{ts2/ts2} disp^{-/-} double mutant discs as it caused in the hh^{ts2/ts2} single mutant. Also, equivalent levels of ptc-lacz expression are induced by Hh-Nu in hh^{ts2/ts2} disp^{-/-} discs and in hh^{ts2/ts2} disp⁺ discs, and punctuate Hh staining was again observed in anterior cells. Thus, unlike Hh-Np, cholesterol-free Hh is neither retained nor compromised in its range of action if produced by disp mutant cells. Since the sole known structural difference between Hh-Np and Hh-Nu is the C-terminal cholesterol moiety, we conclude that it is this lipid anchor that is responsible for the retention of Hh-Np protein by disp mutant cells. From this we infer that the function of Disp is to overcome this retention and thereby permit the release of lipid-modified Hh protein from Hh producing cells.

### Specificity of Disp: GPI-anchored Hh is Not Released From the Cell Surface

Having established that the activity of Disp permits the release of tethered Hh protein, we then addressed the specificity of this release mechanism by asking two questions. First, is Hh activity also dependent on Disp if Hh is tethered by a non-lipid anchor? And second, does Disp also liberate Hh protein if Hh is tethered by a lipid anchor other than cholesterol?

To address the first question, we used a fusion protein (Hh-CD2) in which the signaling domain of Hh is fused to the N-terminus of the type I transmembrane protein CD2 (Strigini and Cohen, 1997). This derivative of Hh has previously been shown to be effectively tethered to expressing cells, and to retain biological activity even in the absence of endogenous Hh (Strigini and Cohen, 1997) We expressed hh-CD2 under en-Ga14 control in a disp^{-/-} hh^{ts2/ts2} mutant background and found that its activity does not depend on the presence of disp. We therefore conclude that Hh protein with a non-lipid tether—like Hh protein with no tether (Hh-Nu)—functions independently of Disp.

Finally, we asked if addition of lipids other than cholesterol would also tether Hh signaling activity, and whether such tethering can be overcome by Disp. We generated a form of Hh-N (Hh-GPI) which carries the C-terminal 54 residues of Drosophila Fasciclin I (Fasl, Zinn et al., 1988) including the glycosyl-phosphatidylinositol (GPI)-anchoring signal of Fasl. As a control we used a derivative of Hh-GPI (Hh-ΔGPI) in which the GPI-anchoring signal was mutated. When Hh-ΔGPI is expressed in marked clones of wing imaginal disc cells, ubiquitous expression of dpp-lacZ is observed in the entire A compartment, which is extended in size. This phenotype is the same as that of Hh-Nu, and indicates that the addition of heterologous sequences does not compromise the long-range signaling activity of Hh-Nu. In sharp contrast, expression of Hh-GPI induces ectopic dpp-lacz expression only in Hh-GPI expressing cells and in their immediate wild-type neighbors. Conversely, wild-type Hh in the same assay induces dpp-lacz in wild-type cells up to five or more cell diameters away. Thus the GPI moiety effectively tethers Hh to the surface of expressing cells. Disp, which is present and active in these cells, can not liberate Hh-GPI as it does Hh-Np, indicating that cholesterol is an important determinant of the Disp-dependent release mechanism of tethered Hh.

### EXAMPLE 1

Identification of dispatched, a novel gene required for Hh signaling

The Drosophila dispatched gene was identified in a genetic screen for new components of the Drosophila Hedgehog signaling pathway. 1737 lethal P-element insertions created by Deak and co-workers (Deak et al., 1997) were recombined onto the FRT80 or FRT82 chromosomes to allow the generation of somatic and germline clones by Flp-mediated mitotic recombination (Chou and Perrimon, 1996; Xu and Rubin, 1993). Recombinants were generated using the eyFLP system (Newsome et al., 2000) to efficiently identify by their mosaic eye color those animals with both the FRT and P[w+] insertions on the same arm. The P element 1(3)S037707 was then identified by screening these lines for hh-like phenotypes. Below we list the genotypes used in our analysis.

disp germline clones y w hsp70-flp; FRT82 disp/FRT82 P[ovo^{D}] ×FRT82 disp/TM3 P [y⁺]

disp clones in adult wings f hsp70-flp; FRT82 disp/M(3R)w124P[f⁺]

hh clones in adult wings y w hsp70-flp; FRT82 hh^{AC}/FRT82 M(3R)w 124 P[y⁺]

disp clones in imaginal discs, using dpp-lacZ or ptc-lacZ y w hsp70-flp; dpp(ptc)-lacZ; FRT82 disp/FRT82 M(3R)w 124 2x P[hsπMyc, w⁺]

disp clones in imaginal discs, hh-lacZ y w hsp70-flp; FRT82 disp hh-lacZ/FRT82 P[hsCD2, y+] hh-lacZ

hh^{ts2} rescue experiments en Ga14/UAS-hh-F(N^{HA}); hh^{ts2}/hh^{ts2} en Ga14/UAS-hh-F(N^{HA}); disp hh^{ts2}/diSp hh^{ts2} en Ga14/UAS-hh-CD2; hh^{ts2}/hh^{ts2} en Ga14/UAS-hh-CD2; disp hh^{ts2}/disp hh^{ts2}

clones expressing Hh, Hh-GPI or Hh-ΔGPI in imaginal discs y w hsp70-flp; dpp-lacZ/UAS-hh (hh-GPI, hh-ΔGPI); actin5>CD2>Ga14 UAS-GFP

hh-temperature sensitive animals were generated using the hh^{ts2} allel (Ma et al., 1993) balanced over the TM6b[Tb]balancer. These animals were allowed to develop at the permissive temperature (18° C.) for 2 to 4 days before shifting to the non-permissive temperature (29° C.). Imaginal discs were dissected and fixed after 3 to 4 days at 29° C.

### Transgenes

Reporter genes used in this study were dpp-lacZ ¹⁰⁶³⁸ (Zecca et al., 1995), hh^{P30} (Lee et al., 1992) and ptc (10.8L) A (Chen and Struhl, 1996).

The UAS-hh transgenes use were derived from the full-length cDNA clone 11 (Lee et al., 1992). UAS-hh-F^{HA} contains the full-length cDNA, whereas UAS-hh-N^{HA} is truncated and contains a stop codon following residue G257, the normal site of Hh autoproteolytic cleavage (Porter et al., 1995). Both transgenes contain a triple HA-epitope tag inserted between hh codons 254 and 255. The UAS-hh-GPI transgene contains sequences of the fasciclin I gene (Zinn et al., 1988) encoding the C-terminal most 54 amino acid (aa) residues fused to Hh at G257. The UAS-hh-ΔGPI harbors the same fusion, but the last 27 residues of Fasciclin I are replaced by a stop codon. The UAS-hh-CD2 transgene was a gift from M. Strigini (Srigini and Cohen, 1997). The tub-disp rescue construct contains full length disp cDNA flanked at its 3′ end by the 3′ UTR of the tubulinal gene. In the UAS-disp transgene, a triple HA tag was inserted in frame at the 3′ end of the open reading frame, followed by the 3′ tubulinα1 UTR. All constructs were inserted into pUAST (Brand and Perrimo, 1993) or into a P element plasmid containing the promoter of the tubulinα1 gene (Basler and Struhl, 1994).

### EXAMPLE 2

Molecular cloning of Drosophila dispatched

Genomic DNA from either side of the P element 1(3)S03770 was obtained by plasmid rescue upon restriction by BamHI or EcoRI. Sequence analysis of rescued fragments revealed that the P element was inserted within sequences of Drosophila Yoyo transposable element, which in turn were flanked on either side by sequences identical to an EST in the Berkley Drosophila Genome Databank (clone No. 1d12634). Thus the P element 1(3)S03770 is inserted within an intron of the disp gene located at position aa812. A 0.8 kb EcoRI to SpeI fragment of this EST clone was used to probe an embryonic 0-8 hour cDNA library. Ten positive clones were picked and sequenced. From this new clones and the original EST clone a composite full length sequence was assembled with an ORF predicting a protein of 1218aa in length. BLAST analysis of the sequence revealed low homology (smallest sum probability (SSP) score of ˜1×10⁸) to mouse NPCI and lower similarity to NCPI (SSP ˜1×10⁶) and Drosophila Ptc (SSP ˜1×10⁵). The highest homology was to a predicted but as yet uncharacterized C. elegans protein (Gen-Bank Acc. No. AAC48001, SSP ˜1×10⁵⁵). Three independent transmembrane (TM) domain prediction programs (TopPred2, TMHMM, HMMTOP) all predicted 12 TM domains in the Disp protein. Mobilization of the original P element insertion resulted in several independent deletions removing sequences C-terminal to the P insertion at position aa 812. All these deletions resulted in the same pupal lethality and small disc phenotype of the original P element induced mutations;

### EXAMPLE 3

Immunoblotting and histochemistry

Protein was prepared from dissected third instar larvae by boiling for 5 minutes in 1×SDS sample buffer (20 larvae/100 μl). Protein samples were run on a 17% acrylamide gel (20 μl wild type; 30 μl dsp^{-/-}), then transferred to nylon membranes. Membranes were blocked, then incubated with mouse α-HA 11 antibody (Babco, 1:1000) followed by a α-HRP 2° antibody (1:10000). Immunoreactive protein were visualized by chemiiluminiscence (ECL, Amersham).

Imaginal discs from third instar larvae were fixed and stained by standard techniques except when using the rabbit a-Hh protein, in which case discs were fixed for 20 min in 4% PFA in PBS. Cell surface staining was assayedas follows: imaginal discs were incubated 30 min in 2% formaldehyde in PBS. Subsequent procedures were the same as with standard preparations. Antibodies were mouse monoclonal α-Ptc (gift from I. Guerrero) , α-CD20X34 (Serotec), α-Hh (gift from P. Ingham and P. Thérond) and α-βGal (Cappel); rat monoclonal α-HA (Boehringer/Roche) and α-DE-Cadherin (gift from H. Oda); and Alexa 488 and 594 fluorescent 2° antibodies (Molecular Probes).

While there are shown and described presently preferred embodiments of the invention, it is to be distinctly understood that the invention is not limited thereto but may be otherwise variously embodied and practiced within the scope of the following claims.

### References

Basler, K., and Struhl, G. (1994). Compartment boundaries and the control of Drosophila limb pattern by hedgehog protein. Nature 368, 208-14.

Beachy, P. A., Cooper, M. K., Young, K. E., von Kessler, D. P., Park, W. J., Hall, T. M., Leahy, D. J., and Porter, J. A. (1997). Multiple roles of cholesterol in hedgehog protein biogenesis and signaling. Cold Spring Harbor Symposia on Quantitative Biology 62, 191-204.

Bellaiche, Y., The, I., and Perrimon, N. (1998). Tout-velu is a Drosophila homologue of the putative tumour suppressor EXT-1 and is needed for Hh diffusion. Nature 394, 85-8.

Brand, A. H., and Perrimon, N. (1993). Targeted gene expression as a means of altering cell fates and generating dominant phenotypes. Development 118, 401-415.

Capdevila, J., and Guerrero, I. (1994). Targeted expression of the signaling molecule decapentaplegic induces pattern duplications and growth alterations in Drosophila wings. EMBO Journal 13, 4459-68.

Carstea, E. D. et al. (1997). Niemann-Pick Cl disease gene: homology to mediators of cholesterol homeostasis. Science 277, 228-31.

Chen, Y., and Struhl, G. (1996). Dual roles for patched in sequestering and transducing Hedgehog. Cell 87, 553-63.

Chen, Y., and Struhl, G. (1998). In vivo evidence that Patched and Smoothened constitute distinct binding and transducing components of a Hedgehog receptor complex. Development 125, 4943-8.

Chou, T. B., and Perrimon, N. (1996). The autosomal FLP-DFS technique for generating germline mosaics in Drosophila melanogaster. Genetics 144, 1673-9.

Couso, J. P., Bishop, S. A., and Martinez Arias, A. (1994). The wingless signalling pathway and the patterning of the wing margin in Drosophila. Development 120, 621-36.

Davidson, E. H. (1990), How embryos work: a comparative view of diverse modes of cell fate specification. Development 108, 365-89

Deàk, P. et al. (1997). P-element insertion alleles of essential genes on the third chromosome of Drosophila melanogaster: correlation of physical and cytogenetic maps in chromosomal region 86E-87F. Genetics 147, 1697-722.

Forbes, A. J., Nakano, Y., Taylor, A. M., and Ingham, P. W. (1993). Genetic analysis of hedgehog signalling in the Drosophila embryo. Development—Supplement, 115-24.

Gil, G., Faust, J. R., Chin, D. J., Goldstein, J. L., and Brown, M. S. (1985). Membrane-bound domain of HMG CoA reductase is required for sterol-enhanced degradation of the enzyme. Cell 41, 249-58.

Golembo, M., Raz, E., and Shilo, B. -Z. (1996). The Drosophila embryonic midline is the site of Spitz processing, and induces activation of the EGF receptor in the ventral ectoderm. Development 122, 3363-3370.

Goodrich, L. V., Johnson, R. L., Milenkovic, L., McMahon, J. A., and Scott, M. P. (1996). Conservation of the hedgehog/patched signaling pathway from flies to mice: induction of a mouse patched gene by Hedgehog. Genes & Development 10, 301-12.

Gurdon, J. B., (1992). The Generation of Diversity and Pattern in Animal Development. Cell 68, 185-99.

Hahn, H., Christiansen, J., Wicking, C., Zaphiropoulos, P. G., Chidambaram, A., Gerrard, B., Vorechovsky, I., Bale, A. E., Toftgard, R., Dean, M., and Wainwright, B. (1996). A mammalian patched homolog is expressed in target tissues of sonic hedgehog and maps to a region associated with developmental abnormalities. J. Biol. Chem. 271, 12125-8.

Hammerschmidt, M., Brook, A., and McMahon, A. P. (1997). The world according to hedgehog. Trends in Genetics 13, 14-21.

Hooper, J. E., and Scott, M. P. (1989). The Drosophila patched gene encodes a putative membrane protein required for segmental patterning. Cell 59, 751-65.

Hua, X., Nohturfft, A., Goldstein, J. L., and Brown, M. S. (1996). Sterol resistance in CHO cells traced to point mutation in SREBP cleavage-activating protein. Cell 87, 415-26.

Ingham, P. W. (1998). Transducing Hedgehog: the story so far. EMBO J. 17, 3505-11.

Jessel T. M and Melton D. A. (1992) Diffusible Factors in Vertebrate Embtyonic Induction. Cell 68, 257-70

Kadowaki, T., Wilder, E., Klingensmith, J., Zachary, K., and Perrimon, N. (1996). The segment polarity gene porcupine encodes a putative multitransmembrane protein involved in Wingless processing. Genes & Development 10, 3116-28.

Lawrence, P. A., and Struhl, G. (1996). Morphogens, compartments, and pattern: lessons from drosophila? Cell 85, 951-61.

Lecuit, T., Brook, W. J., Ng, M., Calleja, M., Sun, H., and Cohen, S. M. (1996). Two distinct mechanisms for long-range patterning by Decapentaplegic in the Drosophila wing. Nature 381, 387-93.

Lee, J. J., Ekker, S. C., von Kessler, D. P., Porter, J. A., Sun, B. I., and Beachy, P. A. (1994). Autoproteolysis in hedgehog protein biogenesis. Science 266, 1528-37.

Lee, J. J., von Kessler, D. P., Parks, S., and Beachy, P. A. (1992). Secretion and localized transcription suggest a role in positional signaling for products of the segmentation gene hedgehog. Cell 71, 33-50.

Liscum, L., and Klansek, J. J. (1998). Niemann-Pick disease type C. Current Opinion In Lipidology 9, 131-5.

Loftus, S. K., Morris, J. A., Carstea, E. D., Gu, J. Z., Cummings, C., Brown, A., Ellison, J., Ohno, K., Rosenfeld, M. A., Tagle, D. A., Pentchev, P. G., and Pavan, W. J. (1997). Murine model of Niemann-Pick C disease: mutation in a cholesterol homeostasis gene. Science 277, 232-5.

Ma, C., Zhou, Y., Beachy, P. A., and Moses, K. (1993). The segment polarity gene hedgehog is required for progression of the morphogenetic furrow in the developing Drosophila eye. Cell 75, 927-38.

Marigo, V., Scott, M. P., Johnson, R. L., Goodrich, L. V., and Tabin, C. J. (1996). Conservation in hedgehog signaling: induction of a chicken patched homolog by Sonic hedgehog in the developing limb. Development 122, 1225-33.

Méthot, N., and Basler, K. (1999). Hedgehog controls limb development by regulating the activities of distinct transcriptional activator and repressor forms of Cubitus interruptus. Cell 96, 819-31.

Mullor, J. L., Calleja, M., Capdevila, J., and Guerrero, I. (1997). Hedgehog activity, independent of decapentaplegic, participates in wing disc patterning. Development 124, 1227-37.

Murone, M., Rosenthal, A., and de Sauvage, F. J. (1999). Sonic hedgehog signaling by the patched-smoothened receptor complex. Current Biology 9, 76-84.

Nakano, Y., Guerrero, I., Hidalgo, A., Taylor, A., Whittle, J. R., and Ingham, P. W. (1989). A protein with several possible membrane-spanning domains encoded by the Drosophila segment polarity gene patched. Nature 341, 508-13.

Nellen, D., Burke, R., Struhl, G., and Basler, K. (1996). Direct and long-range action of a DPP morphogen gradient. Cell 85, 357-68.

Neumann, C., and Cohen, S. (1997). Morphogens and pattern formation. Bioessays 19, 721-9.

Newsome, T. P., Asling, B., and Dickson, B. J. (2000). Analysis of Drosophila photoreceptor axon guidance in eye-specific mosaics. Development, in press.

Nüisslein-Volhard, C., and Wieschaus, E. (1980). Mutations affecting segment number and polarity in Drosophila. Nature 287, 795-801.

Osborne, T. F., and Rosenfeld, J. M. (1998). Related membrane domains in proteins of sterol sensing and cell signaling provide a glimpse of treasures still buried within the dynamic realm of intracellular metabolic regulation. Current Opinion In Lipidology 9, 137-40.

Pepinsky, R. B., Zeng, C., Wen, D., Rayhorn, P., Baker, D. P., Williams, K. P., Bixler, S. A., Ambrose, C. M., Garber, E. A., Miatkowski, K., Taylor, F. R., Wang, E. A., and Galdes, A. (1998). Identification of a palmitic acid-modified form of human Sonic hedgehog. J. Biol. Chem. 273, 14037-45.

Pignoni, F., and Zipursky, S. L. (1997). Induction of Drosophila eye development by decapentaplegic. Development 124, 271-278.

Porter, J. A., Ekker, S. C., Park, W. J., von Kessler, D. P., Young, K. E., Chen, C. H., Ma, Y., Woods, A. S., Cotter, R. J., Koonin, E. V., and Beachy, P. A. (1996a). Hedgehog patterning activity: role of a lipophilic modification mediated by the carboxy-terminal autoprocessing domain. Cell 86, 21-34.

Porter, J. A., von Kessler, D. P., Ekker, S. C., Young, K. E., Lee, J. J., Moses, K., and Beachy, P. A. (1995). The product of hedgehog autoproteolytic cleavage active in local and long-range signalling. Nature 374, 363 6 .

Porter, J. A., Young, K. E., and Beachy, P. A. (1996b). Cholesterol modification of hedgehog signaling proteins in animal development. Science 274, 255-9.

Sanchez-Herrero, E., Couso, J. P., Capdevila, J., and Guerrero, I. (1996). The fu gene discriminates between pathways to control dpp expression in Drosophila imaginal discs. Mech. of Dev. 55, 159-70.

Slusarski, D. C., Motzny, C. K., and Holmgren, R. (1995). Mutations that alter the timing and pattern of cubitus interruptus gene expression in Drosophila melanogaster. Genetics 139, 229-40.

Strigini, M., and Cohen, S. M. (1997). A Hedgehog activity gradient contributes to AP axial patterning of the Drosophila wing. Development 124, 4697-705.

Tabata, T., and Kornberg, T. B. (1994). Hedgehog is a signaling protein with a key role in patterning Drosophila imaginal discs. Cell 76, 89-102.

Tatiana A. Tatusova, Thomas L. Madden (1999), “Blast 2 sequences—a new tool for comparing protein and nucleotide sequences”, FEMS Microbiol Lett. 174:247-250

Taylor, A. M., Nakano, Y., Mohler, J., and Ingham, P. W. (1993). Contrasting distributions of patched and hedgehog proteins in the Drosophila embryo. Mech. Dev. 42, 89-96.

van den Heuvel, M., Harryman-Samos, C., Klingensmith, J., Perrimon, N., and Nusse, R. (1993). Mutations in the segment polarity genes wingless and porcupine impair secretion of the wingless protein. EMBO Journal 12, 5293-302.

von Heijne, G. (1992). Membrane protein structure prediction, hydrophobicity analysis and the positive-inside rule. J. Mol. Biol. 225, 487-494.

Wasserman J. D., and Freeman M. (1998). An autoregulatory cascade of EGF receptor signaling patterns the Drosophila egg. Cell 95, 355-364.

Xu, T., and Rubin, G. M. (1993). Analysis of genetic mosaics in developing and adult Drosophila tissues. Development 117, 1223-1237.

Zecca, M., Basler, K., and Struhl, G. (1995). Sequential organizing activities of engrailed, hedgehog and decapentaplegic in the Drosophila wing. Development 121, 2265-78.

Zinn, K., McAllister, L., and Goodman, C. S. (1988). Sequence analysis and neuronal expression of fasciclin I in grasshopper and Drosophila. Cell 53, 577-87.

1. A purified and/or modified polypeptide comprising a lipid sensing domain, in particular a sterol sensing domain functioning in the release of hedeghog proteins and/or lipid modified proteins.

2. The polypeptide of claim 1 that hydrophilizes a lipid modified protein.

3. The polypeptide of claim 1 which modulates the release of lipid modified proteins from cells.

4. The polypeptide of claim 2 wherein the proteins to be released are sterol modified proteins.

5. The polypeptide of claim 1 which modulates the release of proteins functioning as developmental inducers.

6. The polypeptide of claim 5 which modulates the release of hedgehog proteins.

7. The polypeptide of claim 6 which modulates the release of vertebrate hedgehog proteins.

8. A purified and/or modified dispatched polypeptide comprising an amino acid sequence selected from
a) sequences comprising a stretch of at least 100 contiguous amino acids with a minimal identity percentage of 35%, preferably 45% and more preferably 52% to the amino acid sequence of Table 5 and SEQ ID NO: 1 in a blastp comparison.
b) sequences comprising a stretch of at least 150 contiguous amino acids with a minimal identity percentage of 30%, preferably 37% and more preferably 48% to the amino acid sequence of Table 5 and SEQ ID NO: 1 in a blastp comparison
c) sequences comprising a stretch of at least 200 contiguous amino acids with a minimal identity percentage of 27%, preferably 35% and more preferably 46% to the amino acid sequence of Table 5 and SEQ ID NO: 1 in a blastp comparison

9. The polypeptide of claim 8 that has Hedgehog and/or lipid modified protein releasing ability.

10. A purified and/or modified polypeptide comprising an amino acid sequence which is at least 20% identical to the amino acid sequence of Table 5 and SEQ ID NO:1.

11. The polypeptide of claim 10 wherein the dispatched homologuous amino acid sequence is at least 30%, preferably 40% and more preferably 50% identical to the sequence of Table 5 and SEQ ID NO:1.

12. The polypeptide of claim 10 wherein the dispatched amino acid sequence is identical to the sequence of Table 5 and SEQ ID NO:1.

13. The polypeptide of claim 10 that has Hedgehog and/or lipid modified protein releasing ability.

14. Fragments with a length of at least 100, preferably at least 150 amino acids of the polypeptide sequences of claim 8.

15. The fragments of claim 14 with Hedgehog and/or lipid modified protein releasing ability.

16. A purified and/or modified dispatched polypeptide comprising the amino acid sequence selected from the Table 5 and SEQ ID NO:1 and its homologues comprising proteins whose full length amino acid sequence shows at least
a) a 13% minimal identity with Clustal W for amino acids 1-1218.
b) a 25% minimal identity with Clustal W for amino acids 442-635.
c) a 25% minimal identity with Clustal W for amino acids 718-1218.

17. The dispatched polypeptide of claim 16 wherein the amino acid sequence is at least 20%, preferably 30% and more preferably 40% identical to the sequence of the Table 5 and SEQ ID NO:1.

18. Fragments with a length of at least 100, preferably at least 150 amino acids of the polypeptide sequences of claim 16.

19. The fragments of claim 18 with Hedgehog and/or lipid modified releasing ability.

20. An isolated nucleotide sequence encoding a polypeptide of claim 1.

21. A isolated nucleotide sequence encoding a polypeptide of claim 8.

22. A isolated nucleotide sequence encoding a polypeptide of claim 16.

23. A method for the secretion of lipid modified proteins comprising cultivating suitable host cells transformed or transfected with a nucleic acid encoding a protein of claim 1 in an expression construct enabling the expression of said protein, and isolating the secreted proteins.

24. The method of claim 23 wherein the host cell is a eucaryotic cell.

25. The method of claim 24 wherein the host cell is a mammalian cell.

26. A method for the secretion of lipid modified proteins comprising cultivating suitable host cells transformed or transfected with a nucleic acid encoding a protein of claim 8 in an expression construct enabling the expression of said protein and isolating the secreted proteins.

27. The method of claim 26 wherein the host cell is a eucaryotic cell.

28. The method of claim 27 wherein the host cell is a mammalian cell.

29. A method for the secretion of lipid modified proteins comprising cultivating suitable host cells transformed or transfected with a nucleic acid encoding a protein of claim 16 in an expression construct enabling the expression of said protein, and isolating the secreted proteins.

30. The method of claim 29 wherein the host cell is a eucaryotic cell.

31. The method of claim 30 wherein the host cell is a mammalian cell.

32. A method for screening for agonists or antagonists of dispatched activity comprising contacting a suitable cell containing a dispatched expression construct with a candidate compound and determining the effect of said compounds on the activity and/or amount of lipid modified proteins.

33. A method for screening for agonists or antagonists of dispatched activity wherein eggs or larvae of a fly which is either wildtyp, dispatched negative or carrying a dispatched mutant gene with reduced or enhanced dispatched activity are contacted with a candidate compound and analysing the resulting phenotype.

34. A fly carrying a dispatched mutant gene wherein the mutation causes an enhanced, reduced or a loss of dispatched activity.

35. An antibody capable of binding specifically to a polypeptide of claim 1.

36. An antibody capable of binding specifically to a polypeptide of claim 8.

37. An antibody capable of binding specifically to a polypeptide of claim 16.

38. An isolated and/or modified polypeptide comprising the amino acid sequence of SEQ ID NO:3.

39. An isolated and/or modified polypeptide comprising the amino acid sequence of SEQ ID NO:4.


SEQUENCE LISTING (4 sequences)

SEQ ID NO: 1 - DNA, 3657 bp, Drosophila melanogaster
  CDS (1)..(3657)
atgttgtgcttcgactcggagaggatgaactggtactaccacgtcctggccaggcgtccctacctggtggtcgtctccattgctgtctactgtgtcgcttgcatcatcgtggccctcgtcctgaacaagctgccggacttcagtgatcccaccttgggcttcgaaacccgcggcaccaaaattggagaacgactgacggcctggtacaacctgctgcaagagactgaccaccatggtgctcttttctcaaatccatcggatctgtgggagaggagacgggtagagcagggctatgtggagacgaagctgcatccgaatcacaggcgacgcaaaaacaagcacaagaacagaaacaagaacaagcgacgcaaggaacaaaatcaaagcagccacgagcatcacgatgtggcgcagaagatgatgcagttcaaaaaacggctgaaggctactagttctccctctccaaatctcggctttgacacctggatcggggacagtggagtatttcgcgactacgagattacaaacgacagcgcttcctcctctttggaacccacgcgacgcactgagcaaatcgaatatggccacaacaccacctcagtggacgaggaagagcatcagcagcgggtgcagaccaagaagagcacttggcggctcctaaagcaagccgccaccttgcccaccgacggttgggcagacatgcatcgtcgccaacccatcgagggattcttttgcgattcatcgccccggaaggagtactctcattttgtggtccagcggatcggtcccaacgccaccgattctctatttgatttgaacggactgctggccatgtgtcagttgcaggatcagattaccgaagtccccagctaccgggcattttgcgagccggagatgcttaccaccgagtgctgccggccatggtcactgcccaactacgccgccatgctggccaacaaaagttcctgtttcgaccttaccacggaggatgttacttcgctgcacacgctgctcttgggctgctatgagtacttccatgacctaaagatggacaaccactgcaacgaaataccgcattgtcgtgctcctgaagagtgcaagcggctaaatatcgtctttaacgtcctgaattttctcaccgactttagtttcatcaagtctaatgactcgaatgtatacctgaaatacgccatgatttttatacccgtggcgcaatccaaccgcttgctgccactgttccacgaatgggaagacgtggagctgatcaacgagcttgtggaagtggttgcgatggacttgggcctcgaaaacgaactctttaacgagctgctgctgacggacgtgtggctagtttcgctgggagggacattcgtcatggctagcgtgtggttgtacacgggatcggcattcatcaccttgatgtcctgcgtagccatatgcttttcgctaggactagcgtactttttttacgccatcgtgctggagtttgagttcttcccctatatgaaccttctggccgtggtagtaataattggcattggagcagacgacgtatttctgtttcttaagatctggcactgtgtgctgaccgagaggttcagcaatcgatgtaccttgaccactcagtcccagagtgctcttcccaccctggagaacagtgatcacacggagtcactggaaaacataatggcgctgactatgcgacatgccgctgcctccatgttcgttacctcacttaccaccgccggcgccttttatgcctcctacagcagctctataacagctataaagtgctttgggatttttgcgggaactgtcgtggtgaccaactacttactaatgataacttggttgcctgcatcggtctccatcatggaacgactatttgccacaaggatgtcctgccatcatccgatgtcaataaagctgatccacgcctgcaagaagtcaattaaccgattttgtcagatgtttgaagagtgcatcacgaaaagcatcatgaactatgcctatctctggctgctgatctttggggctctaggcgcgtccagtgccgtcattgtgttctggtatccaggacttcagttgccggaaaaatcacacttccagctctttgtgtcaaagcatccatttgaggtttactccagtctcaagcagcagttctggttcgagaaaccatggcaggcgtacgagaacttcaagatgcacatgcatttcgtctggggcgttcaagcggtggacgacggcgactatacgaaccccaactcatacggccacctgcactatgacaataattttaacgtatccagcaggccggcacaactctggatccttgatttttgccagagtgtgcgccagcaacccttttacaaagagactctcggcatgctgttgcccaattgtttcattgaaaatcttatcgactatatgaagcgcagatgcatcgatgatatggacagtaccaggaaagaccgttcaccctgctgtgacgcacagtttcccttcgagccgcacatatttgagtactgcttgccacaaagcatatccaacatgtatgacactacatttttccggcccggcgtggcaggacccaagtttgcagaggctccccgactggagactgaggattacctaggaatgagtggaaatgagagtgcagagtacagcaccaacggatcatttacaccgctactagtcaaagccctggtcatcgagttcgagtccaacgtggcctacagcaccatctacgcaaatattaggcagttctacgagtctgtagagcactggtttcagatgcagttaaaaacggcaccgccggaacttcaaggaggatggttcacgagcgacctgaaattttacaatgtgcaggacaccctttcgcacgacacctttgttgccatttgtctggccatggctgcatcacttgcagtgctcctatgcttcaccgtaaacatattgatttccatctacgccgtactaaccgtgtcgctaagtatttttaacaccgtggctgtgctcatcctgctcggctggcagcttaacatcttggagagcattgcggtgagcacggctatcggtctggctgtggactttagcctacattacggtattcactaccggatgtccccggttaaggagagattggcagccacacagtttgtactatcccgcatcattggacccacagtgatggcggccaccacaactggtctagctggcggaatcatgatggcatccaacatattgccctacatacagattggcgtcttcctggtcgttgtcatgatcgttagctggttctatgccactttctttctaatgagtctgcttcgggttgccggtcctcagcatggttttctggaactcaagtggccgttgtggagcaagcgaagcagtggcagcagcaagttctacgagcggaaacccagccaagtgatcgccagcgagcagctgctgacccccacaagctcggccatcgttgagttggcgaactcggagacgcacgaactggagtccctaaactccaacagcctgatcaaaaccatttcgggcatcgaaagcgcccacgcattgtcctcgctgccgagggacttcgagcactcattccagacgatgcacgagtgcaaatatcaaacgtatccgtctacatccaattga

SEQ ID NO: 2 - protein, 1218 aa, Drosophila melanogaster
MLCFDSERMNWYYHVLARRPYLVVVSIAVYCVACIIVALVLNKLPDFSDPTLGFETRGTKIGERLTAWYNLLQETDHHGALFSNPSDLWERRRVEQGYVETKLHPNHRRRKNKHKNRNKNKRRKEQNQSSHEHHDVAQKMMQFKKRLKATSSPSPNLGFDTWIGDSGVFRDYEITNDSASSSLEPTRRTEQIEYGHNTTSVDEEEHQQRVQTKKSTWRLLKQAATLPTDGWADMHRRQPIEGFFCDSSPRKEYSHFVVQRIGPNATDSLFDLNGLLAMCQLQDQITEVPSYRAFCEPEMLTTECCRPWSLPNYAAMLANKSSCFDLTTEDVTSLHTLLLGCYEYFHDLKMDNHCNEIPHCRAPEECKRLNIVFNVLNFLTDFSFIKSNDSNVYLKYAMIFIPVAQSNRLLPLFHEWEDVELINELVEVVAMDLGLENELFNELLLTDVWLVSLGGTFVMASVWLYTGSAFITLMSCVAICFSLGLAYFFYAIVLEFEFFPYMNLLAVVVIIGIGADDVFLFLKIWHCVLTERFSNRCTLTTQSQSALPTLENSDHTESLENIMALTMRHAAASMFVTSLTTAGAFYASYSSSITAIKCFGIFAGTVVVTNYLLMITWLPASVSIMERLFATRMSCHHPMSIKLIHACKKSINRFCQMFEECITKSIMNYAYLWLLIFGALGASSAVIVFWYPGLQLPEKSHFQLFVSKHPFEVYSSLKQQFWFEKPWQAYENFKMHMHFVWGVQAVDDGDYTNPNSYGHLHYDNNFNVSSRPAQLWILDFCQSVRQQPFYKETLGMLLPNCFIENLIDYMKRRCIDDMDSTRKDRSPCCDAQFPFEPHIFEYCLPQSISNMYDTTFFRPGVAGPKFAEAPRLETEDYLGMSGNESAEYSTNGSFTPLLVKALVIEFESNVAYSTIYANIRQFYESVEHWFQMQLKTAPPELQGGWFTSDLKFYNVQDTLSHDTFVAICLAMAASLAVLLCFTVNILISIYAVLTVSLSIFNTVAVLILLGWQLNILESIAVSTAIGLAVDFSLHYGIHYRMSPVKERLAATQFVLSRIIGPTVMAATTTGLAGGIMMASNILPYIQIGVFLVVVMIVSWFYATFFLMSLLRVAGPQHGFLELKWPLWSKRSSGSSKFYERKPSQVIASEQLLTPTSSAIVELANSETHELESLNSNSLIKTISGIESAHALSSLPRDFEHSFQTMHECKYQTYPSTSN

SEQ ID NO: 3 - protein, 931 aa, Caenorhabditis elegans
MSTVSRITERIFMKYAHVVIDYPIICIVLTGTISVILTSWALSFNYQVIDFDPTKGFETRGSPLSSARMTLEAMKPHQASNENILRQDPGRRKRYIKNDTTTTLDPITVNYDDYGVDSEPNSSDLEDPCEMYGAIGKALPYDMIEYLGKIMIRVSSYDDLFSLNVMKHLCQIDSIVDNLIIESNYTNPIQALKHSLNIPYYTTCPNMTTQNSCEALNENDILNFRYLLQKCKINSTDEVCSAFSINQVNNWLLTKGNSSDFIIVVVLKVTMWNGAENRDFYDDLIDKLKDHLERNPHTRMAGIALNMKNKVFQERIQTDSLFAAFSALLVFSCFLIYSRSIIFTCIILMVVTLSSGVAFFIYTVVLGIDFFPFINLLVVVILISIGADDAFLLLVYYRREVERMSHLEYKVGSIYIPLYRESDLLSRSLRLSLHHSLVSMFVTSLTTASTFLTNLSSPVIVLRCFGVYAALTVTVNYILVVLILPGAIILSRPIRKKLSRGDEEPEKIESHSYFASKITETTHYFRFGIFICSLIMTGLSLFIIFQNPGLKTPQTNPTKLLVDSNIHEYFDNNVHHFNFQWQRSARLVKNFVFGVDAIKETSTLSPYNKPSKNFSQAHYSLDTDKLDFYRRIVNLESKKYQLVNYTHVSWADKILQANESCFSENKTIIHECILSASVRNKNLIHQFPDDFSVIPGDGPFIDQDLKVVGYFISIPSNQKLQVDTEMIGSFFQEIEESCKQIKNATSDSVLCLSSTEITRFYDIVSQLRSSSFTSVAISLGICLIVIIACTRVIKLSIISSVIIFFVILWTVASLILLGWQLSVVESTILIITIGLSFDYTLHYVVAIRDTKCVPASEKLTSAHSTAGIACVFGSLTLFLAGCPLLFSQTASFYQIGTMLVILGITSLFGASIVLPSFLMVFSCGDRLQSTK

SEQ ID NO: 4 - protein, 757 aa, Homo sapiens
YKKLFMFERVHHGEELHMPITVIWGVSPEDNGNPLNPKSKGKLTLDSSFNIASPASQAWILHFCQKLRNQTFFYQTDEQDFTSCFIETFKQWMENQDCDEPALYPCCSHWSFPYKQEIFELCIKRAIMELERSTGYHLDSKTPGPRFDINDTIRAVVLEFQSTYLFTLAYEKMHQFYKEVDSWISSELSSAPEGLSNGWFVSNLEFYDLQDSLSDGTLIAMGLSVAVAFSVMLLTTWNIIISLYAIISIAGTIFVTVGSLVLLGWELNVLESVTISVAVGLSVDFAVHYGVAYRLAPDPDREGKVIFSLSRVGSAMAMAALTTFVAGAMMMPSTVLAYTQLGTFMMLIMCISWAFATFFFQCMCRCLGPQGTCGQIPLPKKLQCSAFSHALSTSPSDKGQSKTHTINAYHLDPRGPKSELEHEFYELEPLASHSCTAPEKTTYEETHICSEFFNSQAKNLGMPVHAAYNSELSKSTESDTGSALLQPPLEQHTVCHFFSLNQRCSCPDAYKHLNYGPHSCQQMGDCLCHQCSPTTSSFVQIQNGVAPLKATHQAVEGFVHPITHIHHCPCLQGRVKPAGMQNSLPRNFFLHPVQHIQAQEKIGKTNVHSLQRSIEEHLPKMAEPSSFVCRSTGSLLKTCCDPENKQRELCKNRDVSNLESSGGTENKAGGKVELSLSQTDASVNSEHFNQNEPKVLFNHLMGEAGCRSCPNNSQSCGRIVRVKCNSVDCQMPNMEANVPAVLTHSELSGESLLIKTL